(19) United States
(12) Patent Application Publication
(10) Pub. No.: US 2025/0134512 A1
(43) Pub. Date: May 1, 2025

(54) WOUND RETRACTOR WITH MULTI-SEGMENT OUTER RING

(71) Applicant: Applied Medical Resources Corporation, Rancho Santa Margarita, CA (US)

(72) Inventors: Jeremy J. Albrecht, Rancho Santa Margarita, CA (US); Matthew M. Becerra, Lake Forest, CA (US); Eric Nguyen, Rancho Santa Margarita, CA (US)

(21) Appl. No.: 19/010,969

(22) Filed: Jan. 6, 2025

Related U.S. Application Data
(63) Continuation of application No. 18/182,927, filed on Mar. 13, 2023, now Pat. No. 12,185,932.
Application No. 18/182,927 is a continuation of application No. 16/739,796, filed on Jan. 10, 2020, now Pat. No. 11,602,338.
Application No. 16/739,796 is a continuation of application No. 15/288,846, filed on Oct. 7, 2016, now Pat. No. 10,575,840.
(60) Provisional application No. 62/238,608, filed on Oct. 7, 2015.

(51) Int. Cl. A61B 17/02; A61B 17/00; A61B 17/34

(52) U.S. Cl. CPC A61B 17/0293; A61B 17/0218; A61B 17/3431; A61B 2017/00876; A61B 2017/00902; A61B 2017/0225; A61B 2017/3435; A61B 2017/3443; A61B 2017/3492

(57) ABSTRACT

A retractor/protector suitable for use in a surgical incision or a natural orifice comprises a longitudinal axis defining an instrument access channel extending from a proximal end to a distal end; a flexible outer ring; an inner ring; a flexible sheath extending between the outer ring and the inner ring; and at least one rigid segment adapted to attach to the flexible outer ring to thereby increase the rigidity of the outer ring. Embodiments of the retractor/protector are described that have interlocking and non-interlocking rigid segments. Embodiments are also described that have bases that insert into or under the flexible outer ring in addition to or in lieu of rigid segments to increase rigidity and/or provide support for a detachable cap.

### CROSS REFERENCE TO RELATED APPLICATIONS

This application is a continuation of U.S. patent application Ser. No. 18/182,927, filed Mar. 13, 2023, which is a continuation of U.S. patent application Ser. No. 16/739,796, filed Jan. 10, 2020, now issued as U.S. Pat. No. 11,602,338, which is a continuation of U.S. patent application Ser. No. 15/288,846, filed Oct. 7, 2016, now issued as U.S. Pat. No. 10,575,840, which claims the benefit of U.S. Application No. 62/238,608, filed Oct. 7, 2015, the entire disclosure of which is incorporated by reference.

### TECHNICAL FIELD

This invention generally relates to medical devices and, more specifically, to a wound retractors having detachable rigid segments attached to the outer rings of the retractor or protector.

### Discussion of the Relevant Art

Wound retractors/protectors have been described in U.S. Pat. Nos. 7,650,887; 7,727,146; 7,883,461; 7,913,697; 8,235,054, and 8,267,858, the disclosures of which are hereby incorporated by reference in their entireties. The basic components of such wound retractors include an outer ring or rings, an inner ring, and a flexible sheath or sleeve attached at either end to the outer and inner ring.

The outer ring or rings of the wound retractor may be flexible or rigid. In general, flexible outer rings are more comfortable and easier to deploy, while rigid outer rings provide better retraction and, optionally, serve as a platform for attachment of a cap or other similar devices. What is needed, therefore, is a retractor having an outer ring that combines the comfort and ease of deployment of a flexible ring with the retraction and functionality of a rigid ring.

### SUMMARY

A retractor/protector suitable for use in a surgical incision or a natural orifice comprises a flexible outer ring, an inner ring, a flexible sheath extending between the outer ring and the inner ring, a longitudinal axis defining an instrument access channel extending through the flexible sheath from the flexible outer ring at a proximal end to the inner ring at a distal end and a rigid segment adapted to detachably connect to the flexible outer ring to thereby increase the rigidity of the outer ring. In some embodiments there are two rigid segments, while in other embodiments there are more than two rigid segments detachably connected to the outer ring. In some embodiments, the rigid segments are interlockable. In some interlockable examples, the rigid segments may have a boss on one end and a recess on the other end, the recess configured to receive the boss of a contiguous rigid segment.

In another embodiment of the present invention, the rigid attachment is in the form of a ring-shaped rigid base. The ring-shaped rigid base may have an annular groove circumscribed around the outer circumference of the base, the groove configured to receive the outer ring. In this embodiment, the outer ring can be snap fit into the rigid base to provide rigidity when needed.

In still another embodiment of the present invention, the outer ring is detachable from the flexible sheath. In this embodiment, the outer ring comprises a first magnetic strip. The flexible sheath has a second magnetic strip disposed at the proximal end of the sheath, such that the outer ring is detachably connected to the proximal end of the flexible sheath by magnetic attraction between the first magnetic strip and the second magnetic strip. In alternative embodiments, the outer ring comprises a first tube and a second tube, wherein a rigid support ring is disposed within the first lumen and the first magnetic strip is disposed within the second lumen. In some embodiments, a cap may be detachably connected to the outer ring.

### BRIEF DESCRIPTION OF THE DRAWINGS

FIG. 1 illustrates an isometric view of an adjustable wound retractor.

FIG. 2 is a cross-sectional view of a wound retractor deployed in an incision.

FIG. 3A illustrates a front view of a wound retractor having an outer ring system comprising a flexible outer ring with interchangeable rigid segments.

FIG. 3B illustrates a top view of the wound retractor of FIG. 3A.

FIG. 3C illustrates a perspective view of the wound retractor of FIG. 3A.

FIG. 4A illustrates a top view of the outer ring system of FIGS. 3A-C.

FIG. 4B illustrates a right view of the outer ring system of FIGS. 3A-C.

FIG. 5A illustrates an alternative embodiment of the wound retractor of FIGS. 3A-C, shown in front view.

FIG. 5B illustrates a top view of the wound retractor of FIG. 5A.

FIG. 5C illustrates a perspective view of the wound retractor of FIG. 5A.

FIG. 6A illustrates the outer ring system of FIGS. 5A-C, shown in top view.

FIG. 6B illustrates the outer ring system of FIGS. 5A-C, shown in front view.

FIG. 7A illustrates a wound retractor having a flexible outer ring adapted to mount on a rigid base, shown in top view.

FIG. 7B illustrates a cross-sectional view of the wound retractor of FIG. 7A.

FIG. 7C illustrates a perspective view of the wound retractor of FIG. 7A.

FIG. 8A illustrates the outer ring and rigid base of FIGS. 7A-C, shown in top view.

FIG. 8B illustrates the outer ring and rigid base of FIGS. 7A-C, shown in cross-sectional view.

FIG. 8C illustrates the outer ring and rigid base of FIGS. 7A-C, shown in perspective view.

FIG. 9A illustrates a top view of an alternative embodiment of the wound retractor of FIGS. 7A-C.

FIG. 9B illustrates a cross-sectional view of the wound retractor of FIG. 9A.

FIG. 9C illustrates a perspective view of the wound retractor of FIG. 9A.

FIG. 10A illustrates the outer ring and rigid base of FIG. 9, shown in top view.

FIG. 10B illustrates the outer ring and rigid base of FIG. 9, shown in cross-sectional view.

FIG. 10C illustrates the outer ring and rigid base of FIG. 9, shown in perspective view.

FIG. 11A illustrates a wound retractor having an imbedded magnetic strip, shown in top view.

FIG. 11B illustrates a wound retractor having an imbedded magnetic strip, shown in front view.

FIG. 11C illustrates a wound retractor having an imbedded magnetic strip, shown in perspective view.

FIG. 12A illustrates a front view of the wound retractor of FIG. 11 with broken section lines.

FIG. 12B illustrates a cross-sectional view of the wound retractor shown in FIG. 12A.

FIG. 12C illustrates a close-up view of the outer ring of the wound retractor of FIG. 11.

FIG. 13A illustrates an alternative embodiment of the wound retractor of FIG. 11, shown in top view.

FIG. 13B illustrates an alternative embodiment of the wound retractor of FIG. 11, shown in front view.

FIG. 13C illustrates an alternative embodiment of the wound retractor of FIG. 11, shown in perspective view.

FIG. 14A illustrates a front view of the wound retractor of FIG. 13 with broken section lines.

FIG. 14B illustrates a cross-sectional view of the wound retractor of FIG. 14A.

FIG. 14C illustrates a close-up view of the outer rings of the wound retractor of FIG. 13.

### DETAILED DESCRIPTION

Wound retractors/protectors have been described in U.S. Pat. Nos. 7,650,887; 7,727,146; 7,883,461; 7,913,697; 8,235,054, and 8,267,858, and U.S. application Ser. No. 12/873,115, the disclosures of which are hereby incorporated by reference in their entireties.

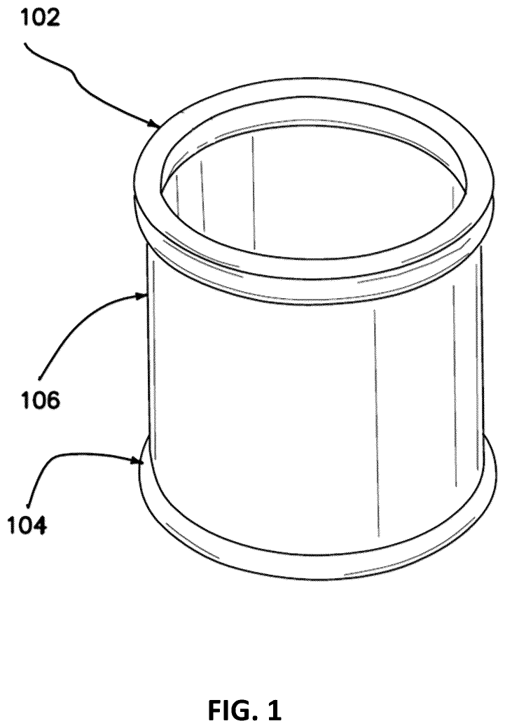

FIG. 1 illustrates an adjustable wound retractor 100 useful in a variety of surgical procedures. The wound retractor 100 includes an outer ring 102, an inner ring 104, and a distensible sheath 106 coupling the outer ring and the inner ring. Outer ring 102 is shown as a single ring, but may also be a double ring or triple ring or multiples thereof. In the illustrated embodiment, the outer ring 102 comprises an annular axis around which the outer ring 102 is rotatable or invertible in a process through which the outer ring 102 is rolled through itself.

To facilitate rolling and to provide for enhanced comfort, the outer ring 102 preferably comprises a flexible material. In some embodiments, the flexible material comprises one or more polymers, for example, flexible engineering plastics. In some embodiments, the flexible material comprises an elastomer, for example, a thermoplastic elastomer. In some embodiments, the outer ring 102 comprises a composite, for example, a polymer and a reinforcing material. Examples of suitable reinforcing materials include fibers, fabrics, and the like, which comprise at least one of polymer, metal, glass, ceramic, and the like. Embodiments of the outer ring 102 are molded and/or extruded as a single piece or as a plurality of pieces that are assembled into the outer ring 102.

In the illustrated embodiment, a cross-sectional shape of the outer ring 102 is generally a figure-8, or first circle and a second circle joined by a web extending therebetween. The outer ring may be solid or may have one or more lumens disposed in the ring. Other embodiments of the outer ring have different cross-sectional shapes, for example, generally oval or elliptical; diamond-shaped or rhomboid; hourglass or dog bone shaped; snowman-shaped; radially flat (washer-shaped outer ring), longitudinally flat (cylindrical outer ring), or flat at another angle (frustoconical outer ring); circular (toroidal outer ring), X-shaped, triangular, square, hexagonal, polygonal, and the like. Some embodiments of the outer ring comprise one or more gripping surfaces that facilitate manually rolling the outer ring around the annular axis thereof. Examples of suitable gripping surfaces include generally flattened surfaces and concave surfaces. Some embodiments of the outer ring 102 have a Möbius configuration in which the outer ring 102 is fabricated with a preloaded circumferential torsional stress, for example, by twisting an elongate member followed by joining the ends.

In some hollow embodiments of the outer ring 102, a wire or rod is disposed in at least one first lumen. Some embodiments of the outer ring 102 do not comprise a rod or wire disposed in a lumen thereof. Some embodiments of a non-compliant outer ring 102 facilitate direct coupling of another device to the outer ring 102 for example, a lid, cap, and/or gel cap. Some embodiments of a compliant outer ring 102 conform to a body surface.

Returning to FIG. 1, the sheath 106 may be coupled to the outer ring 102 and the inner ring 104 by heat seal, adhesive, or other means that are well known in the art. The sheath 106 may be made of a material that is flexible and impermeable to fluids and bacteria.

Embodiments of the sheath comprise sheets, membranes, fibers, and/or strands of one or more materials that endow the sheath with the abrasion and puncture resistance. Suitable sheets, membranes, fibers, and/or strands comprise at least one of natural polymers, semi-synthetic polymers, synthetic polymers, metal, ceramic, glass, carbon fiber, carbon nanotubes, and the like. Suitable natural polymers include cellulose, silk, and the like. Semi-synthetic fibers include nitrocellulose, cellulose acetate, rayon, and the like. Suitable synthetic fibers include polyester, aromatic polyester, polyamide (NYLON®, DACRON®), aramid (KEVLAR®), polyimide, polyolefin, polyethylene (SPECTRA®), polyurethane, polyurea, polyvinyl chloride (PVC), polyvinylidene chloride, polyether amide (PEBAX®), polyether urethane (PELLETHANE®), polyacrylate, polyacrylonitrile, acrylic, polyphenylene sulfide (PPS), polylactic acid (PLA), poly (diimidazopyridinylene-dihydroxyphenylene) (M-5); poly (p-phenylene-2,6-benzobisoxazole) (ZYLON®), liquid crystal polymer fiber (VECTRAN®), and the like, and blends, copolymers, composites, and mixtures thereof. Suitable metals include stainless steel, spring steel, nitinol, super elastic materials, amorphous metal alloys, and the like.

Some embodiments of the sheath material comprises a composite comprising a fabric or textile, for example, at least one of a coated fabric, a laminated fabric, and a fabric embedded in a polymer. Coatings and/or laminations are disposed on one face or both faces of the fabric. Suitable coatings and laminating materials include polymers, for example, at least one of polyurethane, polyether, PVC, polyvinylidene chloride, silicone, styrene-butadiene, polyethylene, polypropylene, ethylene-propylene copolymer, polyisoprene, ethylene vinyl acetate (EVA), ethylene-propylene-diene monomer (EPDM), polyamide (MYLAR®), polyether block amide (PEBAX®), polyether urethane (PELLETHANE®), composites, blends, mixtures, and the like. An example of a suitable composite fabric is polyurethane laminated fabric (PUL). Some embodiments of the coating or lamination modify gas and/or moisture permeability through the sheath material, for example, by controlling the size of pores therethrough. For example, decreasing moisture permeability reduces dehydration of the retracted tissue and/or creates a barrier to pathogens such as bacteria. Increasing gas and moisture permeability permits hydrating and/or oxygenating the retracted tissue. Some materials are selectively permeable to certain fluids. For example, some embodiments of PVC are oxygen permeable and moisture impermeable, thereby permitting simultaneously oxygenating tissue while reducing dehydration. Some embodiments of the coating or lamination comprise an antibacterial or antimicrobial agent. In some embodiments, the antibacterial or antimicrobial agent is a surface agent or is integral to the material. Examples of suitable antibacterial or antimicrobial agents include iodine, antibiotics, silver, triclosan, biocides, and the like. Some embodiments of the coating or lamination provide a smoother and/or lower friction inside surface, which reduces the likelihood of instrument damage to the sheath.

Some embodiments of the sheath comprise a composite comprising a fiber-reinforced polymer film or membrane. Suitable fibers or strands are discussed above. Suitable polymer film materials include at least one of materials discussed above as coating and laminating materials. In some embodiments, the fibers are sandwiched between polymer film layers. In some embodiments, the polymer film layers are independently selected. For example, in some embodiments, the outer layer provides desirable tissue contact properties discussed above, while the inner layer is puncture resistant.

Some embodiments of the sheath comprise a plurality of layers, for example, a fabric layer and a polymer film layer, or a fabric layer sandwiched between polymer film layers. In some embodiments, the layers are secured to each other. In other embodiments, the layers are independent of, or not secured to each other, for example, a polymer film layer and a layer comprising a plurality of strips or bands as discussed above.

Some embodiments of the sheath comprise a fluid-permeable layer disposed on a fluid-impermeable layer, with the fluid-impermeable layer disposed on the inside of the sheath. The fluid-permeable layer contacts the wound margins, thereby permitting a user to supply pressurized fluid and/or apply vacuum to the wound margins. For example, in some embodiments, oxygen, moisture, therapeutic agent, and/or other fluids are supplied to the wound margins. In some embodiments, applying vacuum promotes bleeding, thereby reducing tissue necrosis. Embodiments of the fluid-permeable layer comprise at least one of open cell foam, fabrics, non-woven fabrics, and knit fabrics.

In some embodiments, at least a portion of the sheath is transparent, thereby providing a view of the retracted tissue. In some embodiments comprising a polymer membrane or film, the polymer membrane or film is transparent.

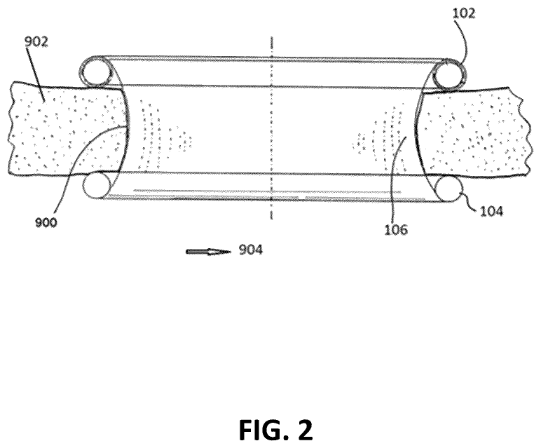

The inner ring 104 may be made of materials of sufficient hardness to retain its shape after insertion into a body cavity 904 (FIG. 2) but sufficiently flexible so as to allow the inner ring to be compressed for insertion through an incision. The materials of which the outer ring 102 is made must allow the outer ring to be turned around its annular axis as further described below. The shape of the outer ring 102 affects both its ability to grip and to provide stability during and after adjustment.

FIG. 2 illustrates the wound retractor deployed in a wound opening 900. To deploy the wound retractor, an incision in the shape of a slit is first made in the body wall 902 of a patient, such as the abdominal wall 902. The inner ring 104 is compressed and the inner ring and sheath 106 are manually inserted into the body cavity 904 through the incision with the outer ring 102 remaining external to the body cavity. Once the inner ring 104 is within the body cavity 904, it expands around the inner surface of the incision 900 so as to be generally parallel to the outer surface of the abdominal wall 902. The sheath 106 provides a working channel from outside the body cavity 904 to inside the body cavity.

The outer ring 102 initially rests above the abdominal wall 902 around the wound opening 900. Since the upper end of the sheath 106 is coupled to the outer ring 102, the sheath 106 can be drawn upwards and radially outward or inward, thereby drawing the inner ring 104 tightly against the inner surface of the abdominal wall 902. Moreover, the intermediate portion of the sheath 106 is drawn tightly against the sides and edges of the wound opening 900, thereby retracting the adjacent tissue and producing a tightly sealed opening in the body cavity 904. The sheath 106 contacts the entire surface of the wound 900 and protectively covers and seals it from contamination and infection. Depending on the size and depth of the incision 900, the user can roll up the sheath 106 by gripping the outer ring 102 and rotating it until the sheath 106 abuts the outer edge of the wound opening 900. The inner ring 104 is adapted for juxtaposition with the inner surface of the abdominal wall 902 and the outer ring 102 is adapted for juxtaposition with the outer surface of the abdominal wall. Both the inner ring 104 and the outer ring 102 are adapted for disposition relative to the incision 900 in the abdominal wall 902. The sheath 106 is adapted to traverse the incision 900 in the abdominal wall 902.

After surgery, the wound retractor 100 may be retrieved by grabbing the inner ring 104 and the sheath 106 and pulling them through the wound opening 900. The use of the sheath 106 and the ease of retracting the outer ring 102 provide higher compression between the inner and outer rings. As a result, the wound retractor 100 provides incremental adjustability to fit a wide range of incision sizes and isolates and protects the wound from bacterial infection as diseased body parts and contaminated instruments are passed through the wound.

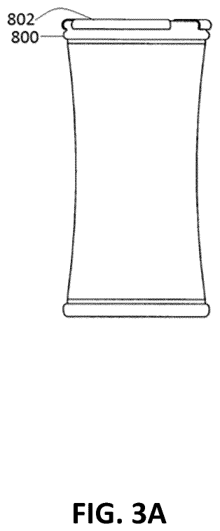

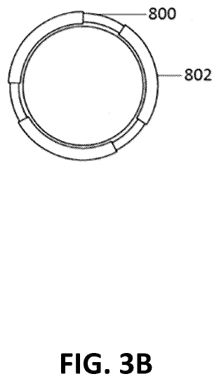

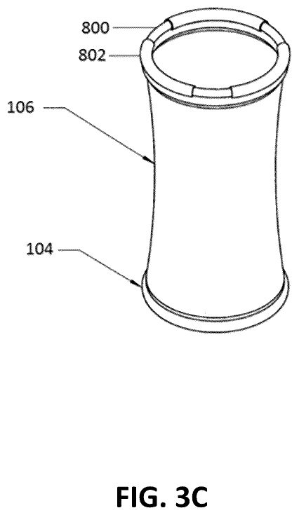

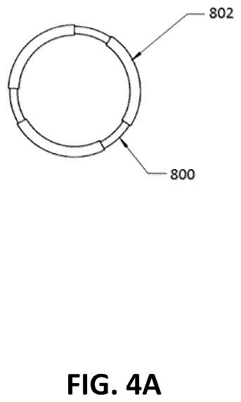

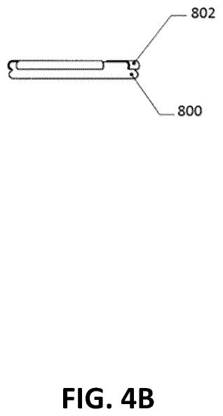

FIGS. 3 and 4 illustrate a new wound retractor, in which the outer ring system is designed to provide the user with both flexibility for ease of deployment and rigidity for full retraction once deployed. The outer ring system comprises a flexible outer ring 800 and one or more interchangeable rigid segments 802 that can be attached to the flexible ring to provide rigidity to the outer ring while in the resting state. The rigid segments can be strategically placed along the retracted wound where, for example, constant maximum visualization is required.

In some embodiments, the rigid segments are designed to snap fit onto the flexible outer ring, and may be partially or continuously placed along the circumference of the flexible ring based on the user's preference and desired degree of rigidity.

FIG. 3A shows a front view of the new wound retractor, in which three rigid segments 802 are snap fit onto the outer ring 800, better seen in top view FIG. 3B or auxiliary view FIG. 3C. The skilled artisan will appreciate that the number and dimensions of the rigid segments will vary depending on intended use. Thus, for example, rigid segments may have a shorter length, in which many more such segments can be attached to the outer ring, or a longer length, in which relatively few segments are attached to provide a more rigid outer ring. Varying the number and dimensions of the rigid segments will effectively modulate the rigidity of the outer ring. Similarly, the segments can be placed anywhere on the outer ring so as to provide different degrees of rigidity. While the rigid segments shown in FIG. 3 are substantially evenly placed around the outer ring, segments can be placed in regions of the outer ring where increased rigidity is desirable while leaving other portions of the outer ring more flexible, depending on the user's particular requirements. A close-up view of the rigid segments 802 attached to the outer ring 800 is shown in FIG. 4A (top view) and FIG. 4B (right view).

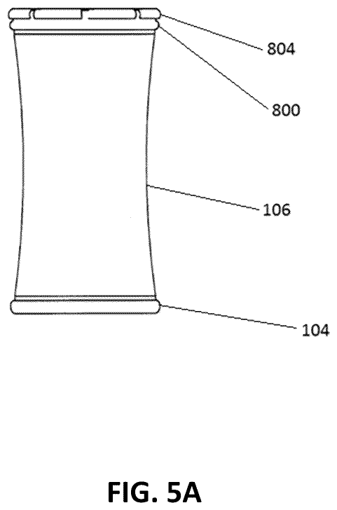

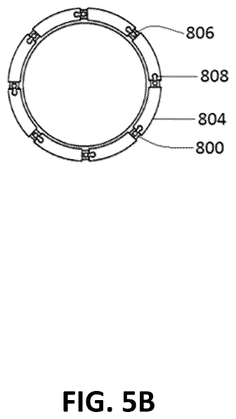

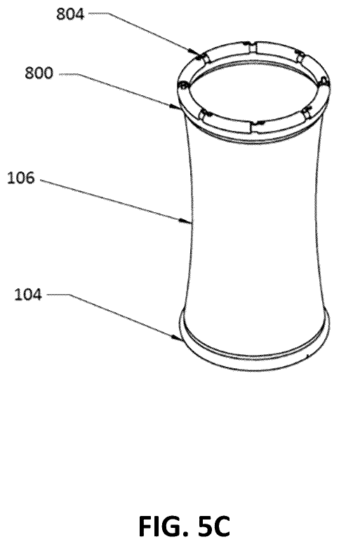

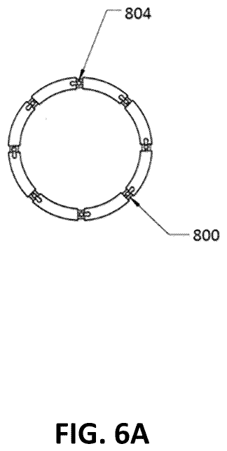

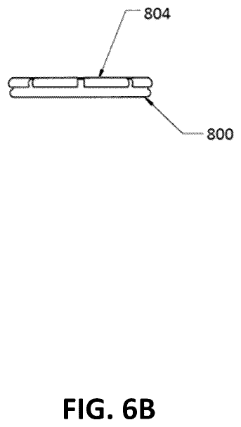

In the embodiment shown in FIGS. 3 and 4, the rigid segments are independent units, not linked together. In an alternative embodiment, as shown in FIGS. 5 and 6, interlockable rigid segments 804 comprise a boss 806 at one end and a recess 808 at the other, the recess adapted to receive the boss of an adjacent rigid segment to thereby link the segments together. In the illustrated embodiment, the interlinked rigid segments form a complete ring, which substantially covers the outer ring 800, best seen in FIG. 5B, 5C and FIG. 6B. It should be appreciated that the boss 806 can be rigid, for easier insertion into the recess 808, or relatively flexible, such that the shape of the interconnected ring of rigid segments 804 can conform to flexible outer rings of different shapes—e.g. circular, oval, elliptical and the like. Other interlocking devices besides a boss and recess are also contemplated in the present invention, including clamps, hooks and other interlocking means known in the art.

It should be appreciated that the outer ring system can be configured to have as many (or as few) rigid segments as needed. The rigid segments can be interconnected, as, for example, with a boss and recess configuration, or may attach to the outer ring independently. It should be further appreciated that providing rigidity to the outer ring with a series of discrete segments accommodates a range of sheath designs, such a circular, elliptical or other non-circular shapes.

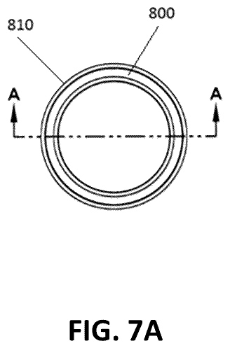

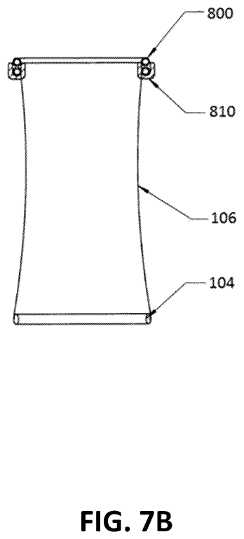

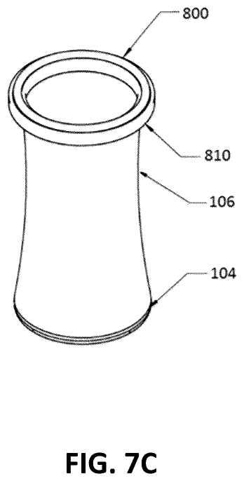

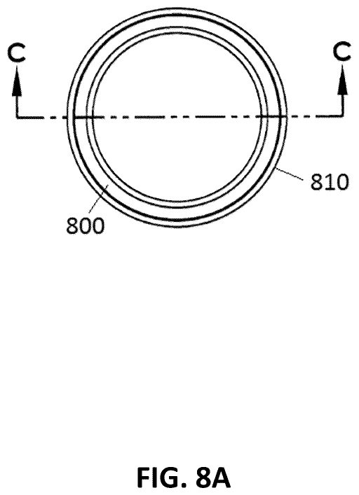

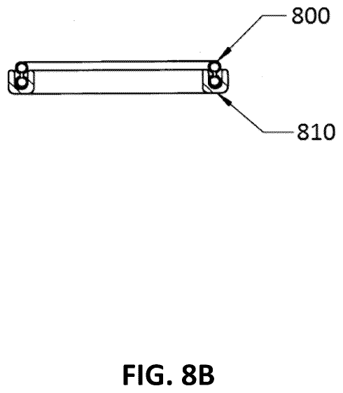

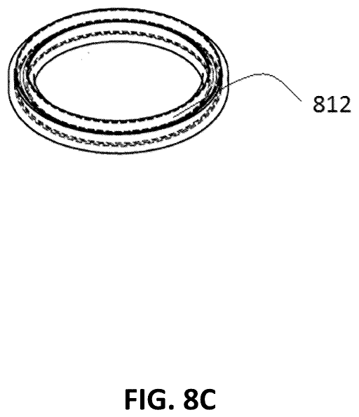

In another embodiment, rigidity may be provided to a flexible outer ring by mounting the ring in a rigid base, allowing the wound to maintain its retracted shape during the duration of the procedure. For example, FIGS. 7 and 8 show a wound retractor in which a ring-shaped rigid base 810 is configured to receive the flexible ring 800 in an annular groove 812 disposed along the proximal or top surface of the base. In use, the retractor is deployed within the incision as described above and the wound retracted; the flexible outer ring 800 is then pulled up through the abdominal base and inserted into the annular groove 812.

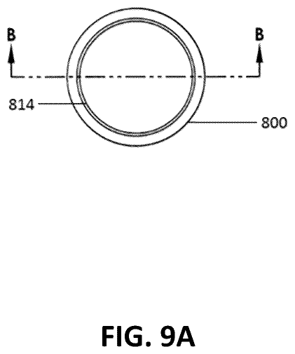

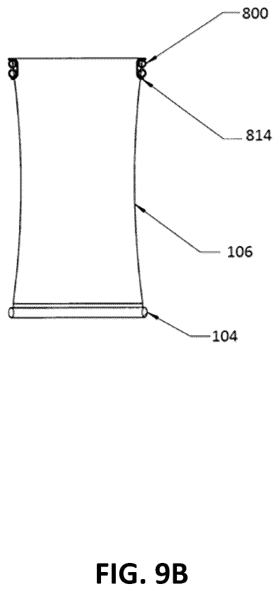

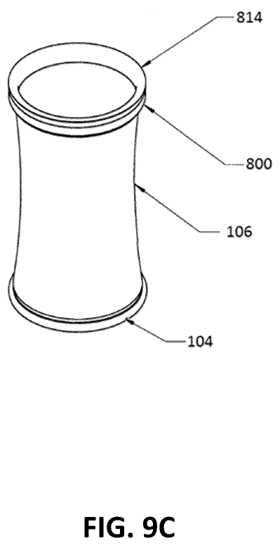

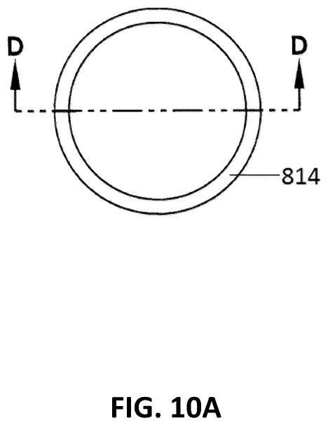

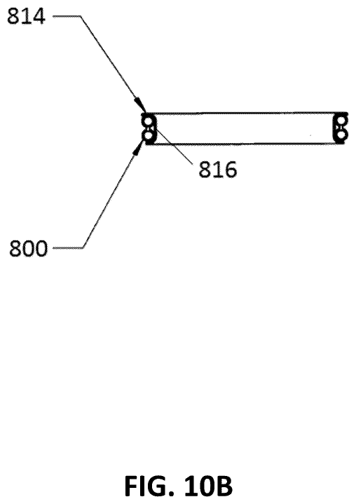

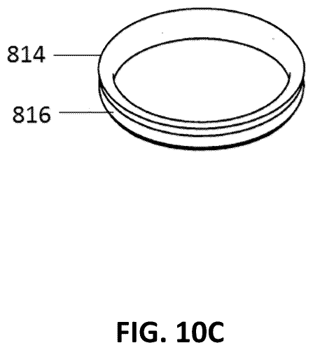

In an alternative embodiment, shown in FIGS. 9 and 10, the rigid base is configured to be deployed inside the flexible outer ring. In this embodiment, the ring-shaped rigid base 814 comprises an annular groove 816 along its outer circumference, adapted to receive the flexible ring 800. After the wound retractor is deployed into the round and the wound is retracted, the rigid base 814 is inserted inside the flexible outer ring 800, which is adapted to snap fit into the annular groove 816 to provide a rigid outer ring system.

The embodiments shown in FIGS. 7-10 are particularly suited to wound retractors adapted for use with a sealing cap. The rigid base, whether mounted under or inside the outer ring, may quickly and easily be added to the flexible outer ring to provide a uniform platform for attachment of a sealing cap. Gel caps suitable for use with wound retractors are described in detail in U.S. Pat. No. 8,267,858, the entire content of which is incorporated herein in its entirety.

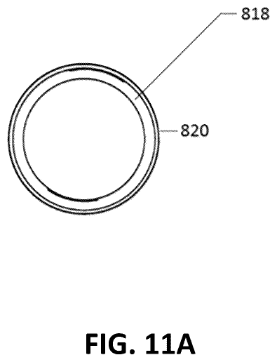

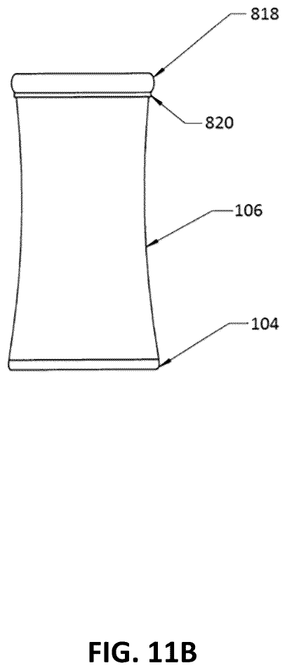

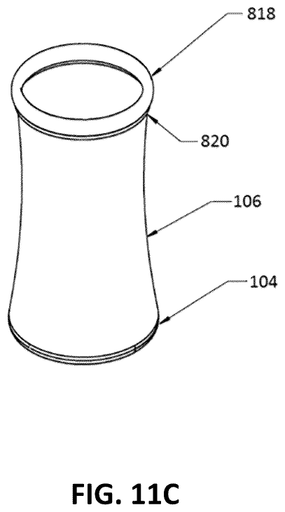

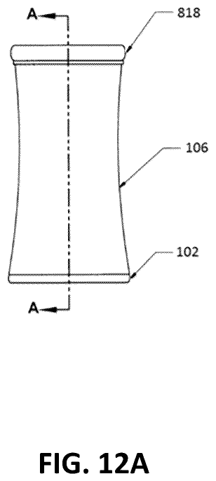

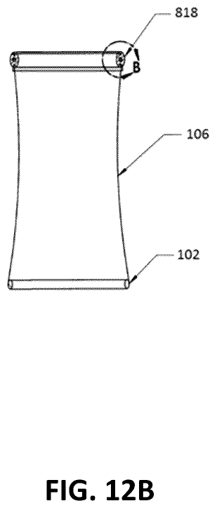

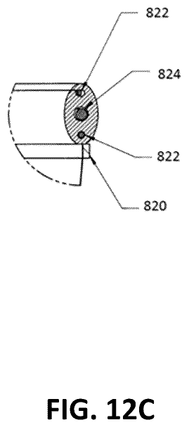

In still another embodiment, shown in FIGS. 11 and 12, the outer ring is detachable from the sheath. In this embodiment, the outer ring 818 attaches to the sheath 106 using magnetic attraction rather than conventional chemical or heat welding. A magnetic ring or strip 820 is imbedded in the proximal end of the sheath, while the outer ring comprises at least one magnetic or ferromagnetic ring 822 that attracts (or is attracted to) the magnetic strip 820. Optionally, a ferromagnetic strip may be imbedded in the proximal end of the sheath, while the outer ring comprises a magnetic ring. Optionally, as best shown in FIG. 12C, the outer ring may also comprise an elastic ring 823 and/or a rigid support ring 824.

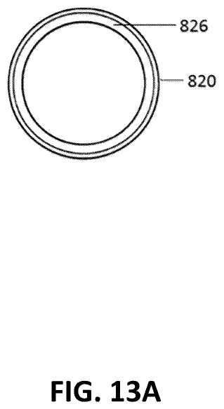

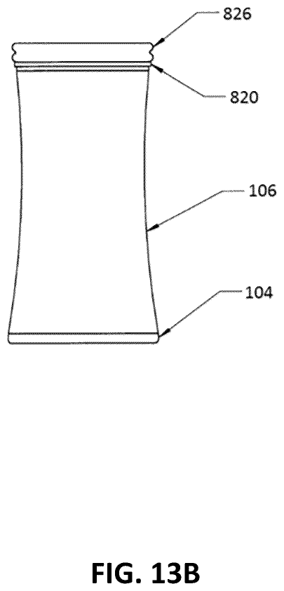

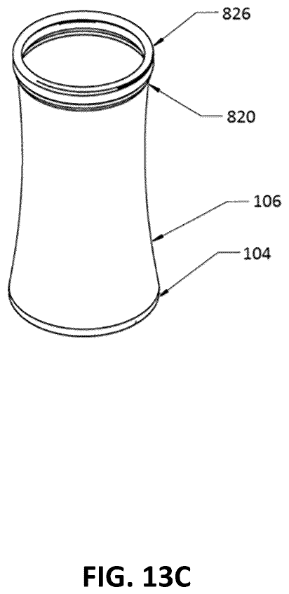

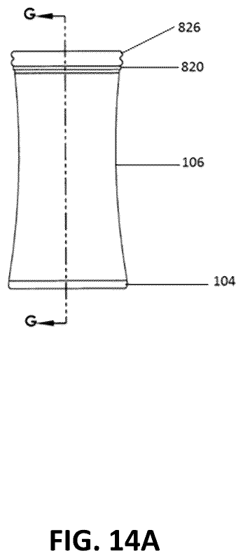

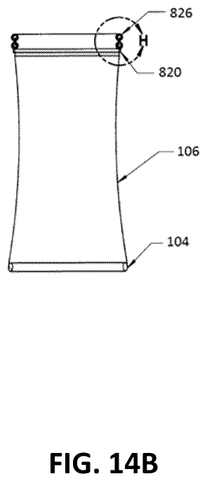

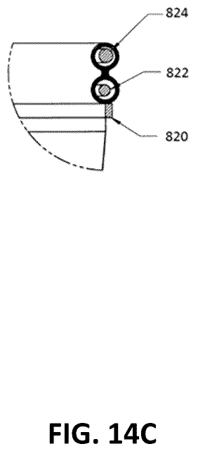

In yet another embodiment, the outer ring can be further configured to accept a sealing cap, such as a gel cap, to provide an instant seal of the body cavity while providing a working channel through the cap into the body cavity. In FIGS. 13 and 14, the wound retractor includes an outer ring 826 comprising two tubes, a first tube and a second tube, each having a lumen (see FIG. 14C). A rigid support ring 824 is disposed in the lumen of the first tube, providing a rigid base for attachment of a cap. A magnetic or ferromagnetic ring 822 is disposed in the lumen of the second tube, to attach the outer ring 826 to the magnetic ring 820 of the sheath 106. Optionally, a ferrous strip may be imbedded in the proximal end of the sheath, while a magnetic ring is disposed within the lumen of the second tube.

While certain embodiments have been particularly shown and described with reference to exemplary embodiments thereof, it will be understood by those of ordinary skill in the art that various changes in form and details may be made therein without departing from the spirit and scope thereof as defined by the following claims.

1. A retractor/protector comprising:
a flexible outer ring;
an inner ring;
a flexible sheath having a proximal end and a distal end, wherein the proximal end is attached to the flexible outer ring, and wherein the distal end is attached to the inner ring;
a longitudinal axis defining an instrument access channel extending through the flexible sheath from the flexible outer ring at a proximal end to the inner ring at a distal end; and
a plurality of rigid segments having a first end and a second end, wherein each of the plurality of rigid segments are adapted to detachably connected to a portion of the flexible outer ring to thereby increase the rigidity of that portion of the outer ring, and wherein each of the plurality of rigid segments are each connected via connectors that interlock the plurality of rigid segments thereby forming a ring.

2. The retractor/protector of claim 1, wherein the connectors comprise a boss, and wherein each of the plurality of rigid segments further comprise a recess at the first and second ends adapted to receive the boss.

3. The retractor/protector of claim 1, wherein the connectors are flexible thereby allowing the ring to adapt to different shapes of the flexible outer ring.

4. The retractor/protector of claim 3, wherein the different shapes of the flexible outer ring comprise one of at least a circular, elliptical, or a non-circular shape.

5. The retractor/protector of claim 1, wherein the connectors comprise clamps.

6. The retractor/protector of claim 1, wherein the connectors comprise hooks.

7. The retractor/protector of claim 1, wherein each of the plurality of rigid segments are adapted to snap fit onto the outer ring.

8. The retractor/protector of claim 1, wherein a length for a number of the plurality of rigid segments making up the ring differ thereby making a rigidity of the outer ring customizable.

9. The retractor/protector of claim 8, wherein the number of the plurality of rigid segments that make up the ring corresponds to the length of each of the plurality of rigid segments.

10. The retractor/protector of claim 9, wherein the number of the plurality of rigid segments that make up the ring further corresponds to dimensions for each of the plurality of rigid segments.

11. The retractor/protector of claim 8, wherein the rigidity of the outer ring corresponds to the number of rigid segments that make up the ring.
